(19) United States
(12) Patent Application Publication
(10) Pub. No.: US 2026/0120888 A1
(43) Pub. Date: Apr. 30, 2026

(54) INFORMATION PROCESSING SYSTEM AND PREDICTION METHOD

(71) Applicant: Hitachi, Ltd., Tokyo (JP)

(72) Inventors: Shunsuke HIDAKA, Tokyo (JP); Wataru TAKEUCHI, Tokyo (JP); Yasuaki NAKAMURA, Tokyo (JP)

(21) Appl. No.: 19/003,478

(22) Filed: Dec. 27, 2024

(30) Foreign Application Priority Data
Mar. 19, 2024 (JP) 2024-043533

(51) Int. Cl. G16H 50/50; G16H 20/00

(52) U.S. Cl. CPC G16H 50/50; G16H 20/00

(57) ABSTRACT

An information processing system, which predicts an unknown binary relation between a treatment method and a biomarker based on a known ternary relation among the treatment method, the biomarker, and a disease, generates for each disease, based on the known ternary relation, a disease-specific bipartite graph that represents the binary relation between the treatment method and the biomarker, calculates, based on the disease-specific bipartite graph, a disease-specific inter-treatment-method similarity between treatment methods, a cross-disease inter-treatment-method similarity between the treatment methods, a disease-specific inter-biomarker similarity between biomarkers, and a cross-disease inter-biomarker similarity between the biomarkers, and calculates and outputs a disease-specific prediction score and a cross-disease prediction score of an unknown edge.

### BACKGROUND OF THE INVENTION

### Claim of Priority

The present application claims priority from Japanese patent application JP 2024-43533 filed on Mar. 19, 2024, the content of which is hereby incorporated by reference into this application.

### 1. FIELD OF THE INVENTION

The present invention relates to an information processing system for predicting an unknown biomarker.

### 2. DESCRIPTION OF RELATED ART

In precision medicine, a biomarker for predicting treatment efficacy and side effects plays an important role in treatment selection, but it is difficult to predict the treatment efficacy and side effects with high accuracy using only knowledge of a known biomarker. Therefore, it is necessary to predict an unknown relation between a treatment method and a biomarker.

In the background art of this technical field, there is a method of constructing a graph based on a known relation and predicting an edge not present in the original graph, that is, an unknown relation. For example, PTL 1 (CN109033754B specification) discloses a method and an apparatus for predicting a disease-associated LncRNA based on a dichotomous network, and the method includes a step of constructing the dichotomous network based on a disease and an LncRNA according to a data set of a known association between the LncRNA and the disease, a step of calculating a disease similarity I and an LncRNA similarity I based on a shared neighbor, a step of calculating a disease similarity II and an LncRNA similarity II based on a SimRank similarity, a step of acquiring an extended disease similarity and an extended LncRNA similarity, a step of refluxing the extended disease similarity and the extended LncRNA similarity to binary networks, and a step of calculating a degree of association between the disease and the LncRNA.

### CITATION LIST

### Patent Literature

PTL 1: CN109033754B specification

### SUMMARY OF THE INVENTION

In the prediction of the unknown relation between the treatment method and the biomarker, the biomarker relates to not only the treatment method but also the disease, and thus a false relation may be predicted and prediction accuracy may decrease when predicting the relation between the treatment method and the biomarker without considering the disease. Therefore, it is necessary to predict the unknown relation between the treatment method and the biomarker with high accuracy in consideration of three types of information, that is, the biomarker, the treatment method, and the disease simultaneously. However, in the technique disclosed in PTL 1, in order to predict the unknown relation between the disease and the lncRNA, a bipartite graph having a disease part and an lncRNA part is generated, a disease inter-node similarity and lncRNA an inter-node similarity are calculated, and an unknown edge is predicted based on the calculated similarities, but no consideration is given to handling the three types of information simultaneously.

An object of the invention is to predict an unknown relation between a treatment method and a biomarker with high accuracy in consideration of a disease.

A representative example of the invention disclosed in the present application is as follows. That is, an information processing system for predicting an unknown binary relation between a treatment method and a biomarker based on a known ternary relation among the treatment method, the biomarker, and a disease includes a computer including a computing apparatus that executes predetermined processing and a storage device connected to the computing apparatus, in which the information processing system further includes: a bipartite graph generation unit that causes the computing apparatus to generate for each disease, based on the known ternary relation, a disease-specific bipartite graph that represents the binary relation between the treatment method and the biomarker; an inter-node similarity calculation unit that causes the computing apparatus to calculate, based on the disease-specific bipartite graph, a disease-specific inter-treatment-method similarity between treatment methods for the each disease, a cross-disease inter-treatment-method similarity between the treatment methods across all diseases, a disease-specific inter-biomarker similarity between biomarkers for the each disease, and a cross-disease inter-biomarker similarity between the biomarkers across the all diseases; an unknown edge prediction unit that causes the computing apparatus to calculate at least one of a disease-specific prediction score and a cross-disease prediction score of an unknown edge using the disease-specific bipartite graph, the disease-specific inter-treatment-method similarity, the cross-disease inter-treatment-method similarity, the disease-specific inter-biomarker similarity, and the cross-disease inter-biomarker similarity; and an output unit that causes the computing apparatus to output at least one of the disease-specific inter-treatment-method similarity, the cross-disease inter-treatment-method similarity, the disease-specific inter-biomarker similarity, the cross-disease inter-biomarker similarity, the disease-specific prediction score, and the cross-disease prediction score.

According to an aspect of the invention, it is possible to predict an unknown relation between a treatment method and a biomarker with high accuracy. Problems, configurations, and effects other than those described above will become apparent in the following description of the embodiment of the invention.

### BRIEF DESCRIPTION OF THE DRAWINGS

FIG. 1 is a block diagram showing a configuration of an information processing system in a first embodiment of the invention.

FIG. 2 shows a configuration of entity set data in the first embodiment of the invention.

FIG. 3 shows a configuration of ternary relation data in the first embodiment of the invention.

FIG. 4 is a flowchart showing bipartite graph generation processing in the first embodiment of the invention.

FIG. 5 shows a disease-specific bipartite graph of a disease entity dᵢ in the first embodiment of the invention.

FIG. 6 shows a disease-specific adjacency matrix G⁽ⁱ⁾ in the first embodiment of the invention.

FIG. 7 is a flowchart showing inter-node similarity calculation processing in the first embodiment of the invention.

FIG. 8 shows a disease-specific inter-treatment-method similarity matrix T⁽ⁱ⁾ in the first embodiment of the invention.

FIG. 9 shows a disease-specific inter-biomarker similarity matrix B⁽ⁱ⁾ in the first embodiment of the invention.

FIG. 10 is a flowchart showing unknown edge prediction processing in the first embodiment of the invention.

FIG. 11 is a flowchart showing output processing in the first embodiment of the invention.

FIG. 12 shows an example of an operation screen for displaying an output result in the first embodiment of the invention in a table format.

FIG. 13 shows an example of an operation screen for displaying the output result in the first embodiment of the invention in a bipartite graph format.

FIG. 14 shows an effect of the embodiment of the invention.

### DESCRIPTION OF EMBODIMENTS

Hereinafter, an embodiment according to the invention will be described with reference to the drawings.

### First Embodiment

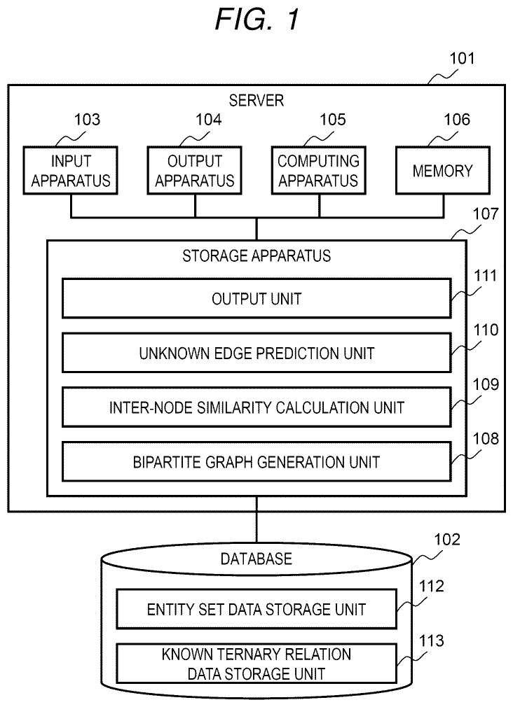

FIG. 1 is a block diagram showing a configuration of an information processing system in a first embodiment of the invention.

The information processing system in the embodiment includes a server 101 and a database 102. The server 101 and the database 102 are connected such that the server 101 can access data stored in the database 102.

The server 101 is a computer including an input apparatus 103, an output apparatus 104, a computing apparatus 105 that executes a program, a memory 106 that stores the program, and a storage apparatus 107.

The input apparatus 103 is a mouse and a keyboard, a touch panel, or the like, and is an interface that receives an input to the server 101. The output apparatus 104 is a display apparatus, a printer, or the like, and outputs a computation result of the computing apparatus 105 in a format visible to a user. A terminal (not shown) connected to the server 101 via a network may function as the input apparatus 103 and the output apparatus 104. In this case, the server 101 may have the function of a web server, and the terminal may access the server 101 using a predetermined protocol (for example, http).

The computing apparatus 105 is a computing apparatus such as a CPU and a GPU, and executes a program loaded in the memory 106. By executing various programs by the computing apparatus 105, each functional unit (for example, a bipartite graph generation unit 108, an inter-node similarity calculation unit 109, an unknown edge prediction unit 110, and an output unit 111) of the server 101 is implemented. The computing apparatus 105 may include a hardware computing apparatus (for example, an ASIC or an FPGA).

The memory 106 includes a ROM that is a non-volatile storage element and a RAM that is a volatile storage element. The ROM stores an immutable program (for example, BIOS). The RAM is a high-speed and volatile storage element such as a dynamic random access memory (DRAM), and temporarily stores a program stored in the storage apparatus 107 and data used when the program is executed.

The storage apparatus 107 is a non-volatile storage apparatus such as a magnetic storage apparatus (HDD) and a flash memory (SSD), and stores the program executed by the computing apparatus 105 and data used when the program is executed. Specifically, the storage apparatus 107 stores a program for implementing each unit of the bipartite graph generation unit 108, the inter-node similarity calculation unit 109, the unknown edge prediction unit 110, and the output unit 111.

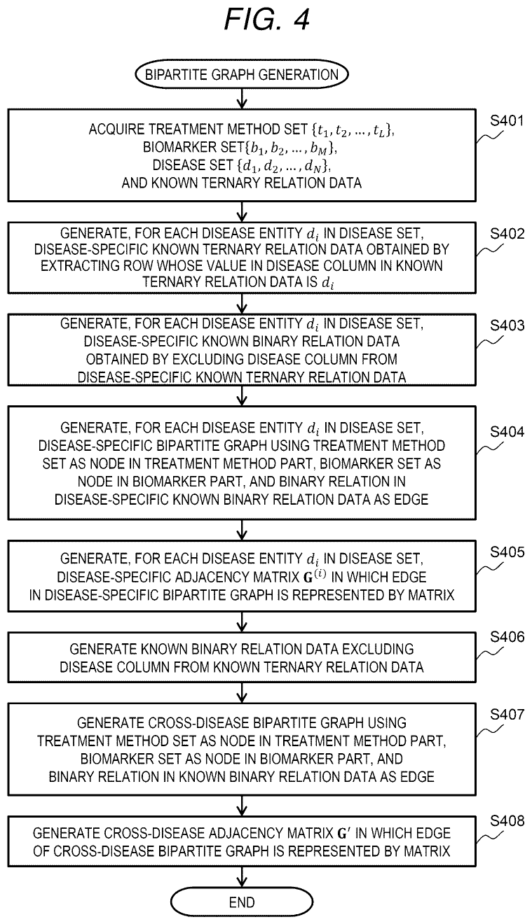

By executing a predetermined program, the bipartite graph generation unit 108 generates a disease-specific adjacency matrix G⁽ⁱ⁾ that represents edges of a bipartite graph of treatment method entities and biomarker entities for each disease entity dᵢ, and generates a cross-disease adjacency matrix G′ that represents edges of a bipartite graph of the treatment method entities and the biomarker entities, which ignores disease information, using entity set data stored in an entity set data storage unit 112 and known ternary relation data 300 stored in a known ternary relation data storage unit 113 (see FIG. 4).

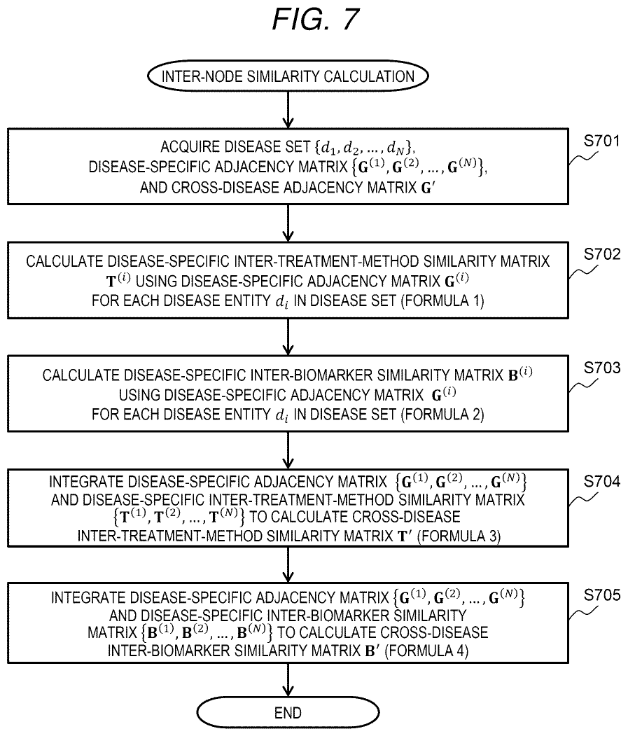

By executing a predetermined program, the inter-node similarity calculation unit 109 calculates, using the disease-specific adjacency matrix G⁽ⁱ⁾ and the cross-disease adjacency matrix G′, a disease-specific inter-treatment-method similarity matrix T⁽ⁱ⁾, a disease-specific inter-biomarker similarity matrix B⁽ⁱ⁾, a cross-disease inter-treatment-method similarity matrix T′, and a cross-disease inter-biomarker similarity matrix B′ (see FIG. 7).

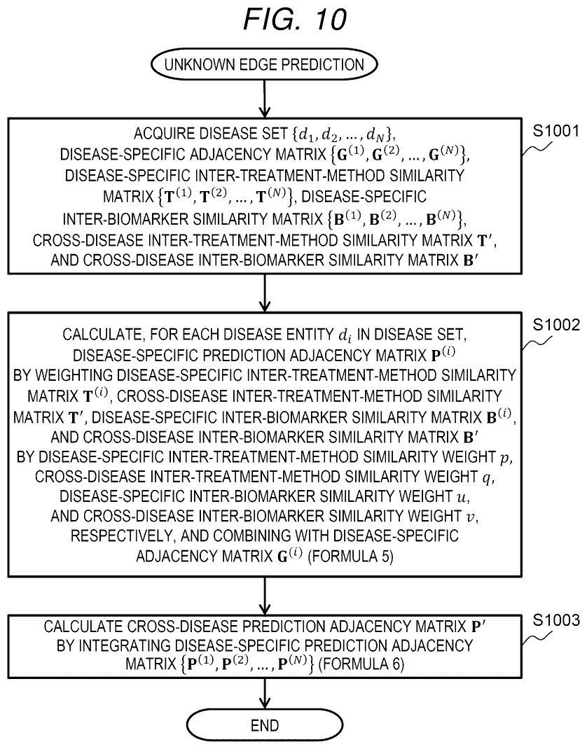

By executing a predetermined program, the unknown edge prediction unit 110 calculates a disease-specific prediction adjacency matrix P⁽ⁱ⁾ and a cross-disease prediction adjacency matrix P′ using the disease-specific adjacency matrix G⁽ⁱ⁾, the disease-specific inter-treatment-method similarity matrix T⁽ⁱ⁾, the disease-specific inter-biomarker similarity matrix B⁽ⁱ⁾, the cross-disease inter-treatment-method similarity matrix T′, and the cross-disease inter-biomarker similarity matrix B′ (see FIG. 10).

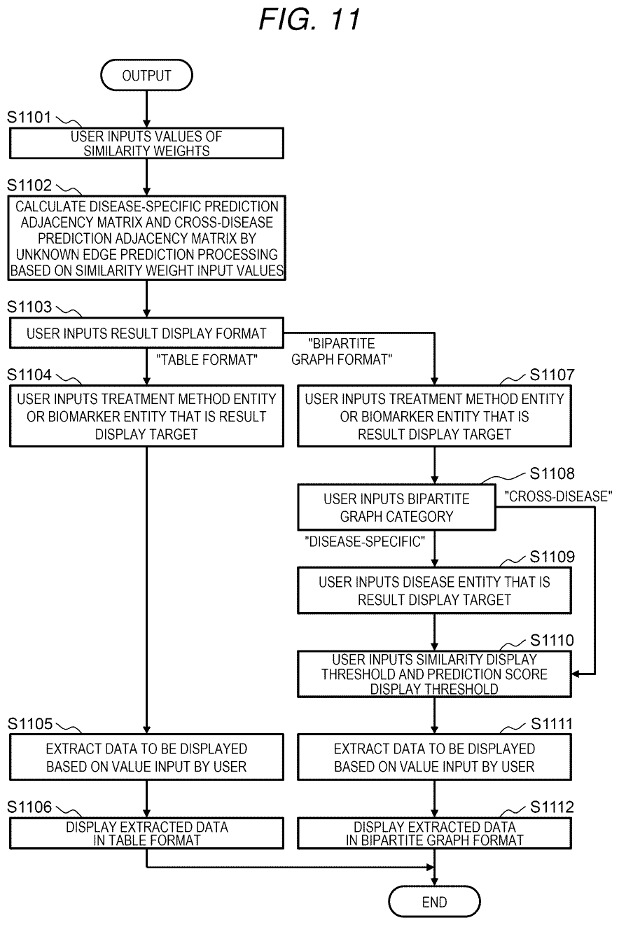

By executing a predetermined program, the output unit 111 visualizes and outputs a calculation result according to an input from the user (see FIG. 11).

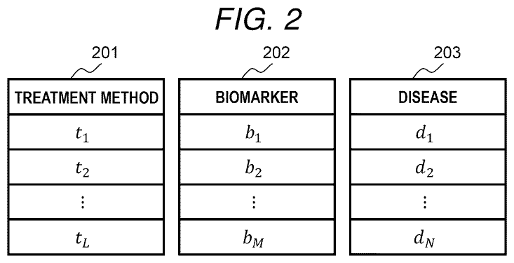

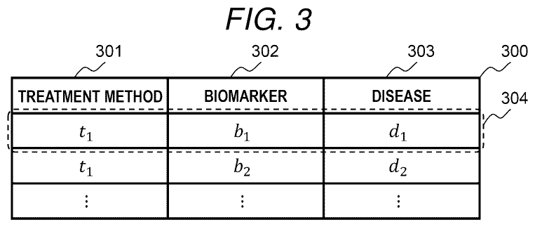

The database 102 includes the storage unit 112 (see FIG. 2) for data to be analyzed by the server 101, that is, entity set data, and the known ternary relation data storage unit 113 (see FIG. 3).

The program executed by the computing apparatus 105 is provided to the server 101 via a removable medium (CD-ROM, flash memory, or the like) or a network, and is stored in the storage apparatus 107 that is a non-transitory storage medium. Therefore, the server 101 may include an interface for reading data from the removable medium.

The server 101 is a computer system implemented on one physical computer or a plurality of computers implemented logically or physically, and may operate on a virtual computer configured on a plurality of physical computer resources. For example, each functional unit may operate on a separate physical or logical computer, or a combination of a plurality of functional units may operate on one physical or logical computer.

FIG. 2 shows a configuration of the entity set data stored in the entity set data storage unit 112 in the first embodiment of the invention.

The entity set data includes data of each of a treatment method set 201, a biomarker set 202, and a disease set 203.

The treatment method set 201 is a set {t₁, t₂, . . . , t_{L}} of L treatment method entities. Each of the treatment method entities is a drug, a surgery, a radiation therapy, or the like. The treatment method entity may be a specific name such as “Ipilimumab” or an abstract name such as “chemotherapy”. The definition of the treatment method entity may be in a format other than the above examples.

The biomarker set 202 is a set {b₁, b₂, . . . , b_{M}} of M biomarker entities. Each of the biomarker entities is a protein, a gene, RNA, a clinical test value, tumor mutation burden (TMB), gut microbiota, or the like. The biomarker entity may be a name such as “ERBB2” or a state such as “ERBB2 overexpression”. The definition of the biomarker entity may be in a format other than the above examples.

The disease set 203 is a set {d₁, d₂, . . . , d_{N}} of N disease entities. Each disease entity is a disease name (for example, “lung cancer”). The definition of the disease entity may be in a format other than the above example.

FIG. 3 shows a configuration of the ternary relation data 300 stored in the known ternary relation data storage unit 113 in the first embodiment of the invention.

The ternary relation data 300 may have columns of a treatment method 301, a biomarker 302, and a disease 303, and may be represented by data in a table format.

Each row in the ternary relation data 300 represents a ternary relation that is known relevance among the treatment method entity, the biomarker entity, and the disease entity. For example, a first row 304 represents that (t₁, b₁, d₁) has a known ternary relation.

FIG. 4 is a flowchart showing bipartite graph generation processing in the first embodiment of the invention. The bipartite graph generation processing shown in FIG. 4 is executed by the bipartite graph generation unit 108 of the server 101.

Step S401: the bipartite graph generation unit 108 acquires the treatment method set {t₁, t₂, . . . , t_{L}} 201, the biomarker set {b₁, b₂, . . . b_{M}} 202, the disease set {d₁, d₂, . . . , d_{N}} 203, and the known ternary relation data 300 (S401). The treatment method set 201, the biomarker set 202, and the disease set 203 are acquired from the entity set data storage unit 112, and the known ternary relation data 300 is acquired from the known ternary relation data storage unit 113.

Step S402: the bipartite graph generation unit 108 generates, for each disease entity dᵢ in the disease set 203, disease-specific known ternary relation data obtained by extracting a row having a value dᵢ in the column of the disease 303 in the known ternary relation data 300.

Step S403: the bipartite graph generation unit 108 generates, for each disease entity dᵢ in the disease set 203, disease-specific known binary relation data obtained by excluding a disease column from the disease-specific known ternary relation data.

Step S404: the bipartite graph generation unit 108 generates, for each disease entity dᵢ in the disease set 203, a disease-specific bipartite graph including a treatment method part 501 and a biomarker part 502, using the treatment method set 201 as a node 504 in the treatment method part 501, the biomarker set 202 as a node 505 in the biomarker part 502, and a binary relation between the treatment method entity and the biomarker entity in the disease-specific known binary relation data as an edge.

Step S405: the bipartite graph generation unit 108 generates, for each disease entity dᵢ in the disease set 203, the disease-specific adjacency matrix G⁽ⁱ⁾ in which the disease-specific bipartite graph is represented by a matrix.

Step S406: the bipartite graph generation unit 108 generates the known binary relation data excluding the disease column from the known ternary relation data.

Step S407: the bipartite graph generation unit 108 generates a cross-disease bipartite graph including the treatment method part 501 and the biomarker part 502, using the treatment method set 201 as the node 504 in the treatment method part 501, the biomarker set 202 as the node 505 in the biomarker part 502, and the binary relation between the treatment method entity and the biomarker entity in the known binary relation data as the edge.

Step S408: the bipartite graph generation unit 108 generates the cross-disease adjacency matrix G′ in which the cross-disease bipartite graph is represented by a matrix.

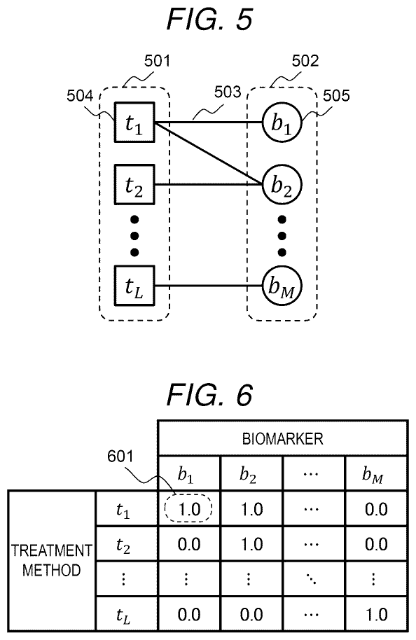

FIG. 5 shows the disease-specific bipartite graph of the disease entity dᵢ generated in step S404 in the bipartite graph generation processing shown in FIG. 4.

The treatment method part 501 represents each treatment method entity in the treatment method set 201 shown in FIG. 2 by the node 504. The biomarker part 502 represents each biomarker entity in the biomarker set 202 shown in FIG. 2 by the node 505. An edge 503 between the treatment method and the biomarker indicates that a treatment method entity t₁ represented by the node 504 and a biomarker entity b₁ represented by the node 505 are contained in the disease-specific known binary relation data of the disease entity dᵢ generated in step S403 in FIG. 4.

The cross-disease bipartite graph generated in step S407 has the same structure as that of the disease-specific bipartite graph shown in FIG. 5, and an only difference therebetween is the edge.

FIG. 6 shows the disease-specific adjacency matrix G⁽ⁱ⁾ that represents the edge in the disease-specific bipartite graph for the disease entity dᵢ shown in FIG. 5.

The disease-specific adjacency matrix G⁽ⁱ⁾ is an L-row M-column matrix whose rows correspond to the treatment method entities in the treatment method set 201 shown in FIG. 2 and whose columns correspond to the biomarker entities in the biomarker set 202 shown in FIG. 2.

A value G⁽ⁱ⁾ⱼₖ of a component in the j-th row and the k-th column indicates whether a treatment method entity tⱼ and a biomarker entity bₖ are contained in the disease-specific known binary relation data of the disease entity dᵢ. A case where the value G⁽ⁱ⁾ⱼₖ is 1 indicates that there is a known binary relation, and a case where the value G⁽ⁱ⁾ⱼₖ is 0 indicates that there is no known binary relation.

Since a value in the first row and the first column in the disease-specific adjacency matrix G⁽ⁱ⁾ is 1, there is a known binary relation between the treatment method entity t₁ and the biomarker entity b₁ for the disease entity dᵢ.

A data structure of the cross-disease adjacency matrix G′ generated in step S408 is the same as a data structure of the disease-specific adjacency matrix G⁽ⁱ⁾ shown in FIG. 5, and only component values are different.

FIG. 7 is a flowchart showing inter-node similarity calculation processing in the first embodiment of the invention. The inter-node similarity calculation processing shown in FIG. 7 is executed by the inter-node similarity calculation unit 109 of the server 101.

Step S701: the inter-node similarity calculation unit 109 acquires the disease set {d₁, d₂, . . . , d_{N}} 203, a disease-specific adjacency matrix {G⁽¹⁾, G⁽²⁾, . . . , G^{(N)}}, and the cross-disease adjacency matrix G′. The disease set 203 is acquired from the entity set data storage unit 112. The disease-specific adjacency matrix and the cross-disease adjacency matrix are generated by the disease-specific bipartite graph generation processing.

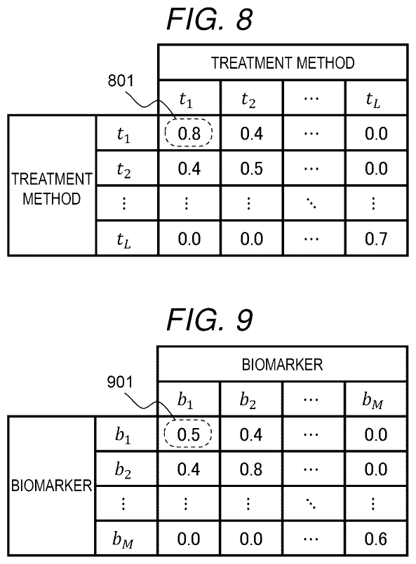

Step S702: the inter-node similarity calculation unit 109 calculates the disease-specific inter-treatment-method similarity matrix T⁽ⁱ⁾ using the disease-specific adjacency matrix G⁽ⁱ⁾ for each disease entity dᵢ in the disease set 203. The disease-specific inter-treatment-method similarity matrix T⁽ⁱ⁾ is an L-row L-column matrix whose rows and columns both correspond to the treatment method entities in the treatment method set 201 shown in FIG. 2, and a value T⁽ⁱ⁾ⱼₖ of a component in the j-th row and the k-th column represents a disease-specific inter-treatment-method similarity between treatment method entities tⱼ and tₖ for the disease entity dᵢ (see FIG. 8).

$${T}_{j k}^{( i )} = \frac{1}{\sqrt{{f}_{1}^{( i )} ( {t}_{j} ) \cdot {f}_{1}^{( i )} ( {t}_{k} )}} {\sum }_{l = 1}^{M} \frac{{G}_{j l}^{( i )} \cdot {G}_{k l}^{( i )}}{{f}_{2}^{( i )} ( {b}_{l} )} \qquad Math 1$$

$${f}_{1}^{( i )} ( {t}_{j} ) := max \{ {\sum }_{k = 1}^{M} {G}_{j k}^{( i )} , 1 \}$$

$${f}_{2}^{( i )} ( {b}_{k} ) := max \{ {\sum }_{j = 1}^{L} {G}_{j k}^{( i )} , 1 \}$$

Formula 1 is a formula for calculating the disease-specific inter-treatment-method similarity T⁽ⁱ⁾ⱼₖ between the treatment method entities tⱼ and tₖ for the disease entity dᵢ. The value of G⁽ⁱ⁾ⱼₖ in Formula 1 is a value of the component in the j-th row and the k-th column in the disease-specific adjacency matrix G⁽ⁱ⁾ for the disease entity dᵢ. A value of f⁽ⁱ⁾₁(tⱼ) in Formula 1 is a node degree of the treatment method entity tⱼ in the disease-specific bipartite graph of the disease entity dᵢ, that is, the number of edges connected to a node. A value of f⁽ⁱ⁾₂(bₖ) in Formula 1 is a node degree of the biomarker entity bₖ in the disease-specific bipartite graph of the disease entity dᵢ. In Formula 1, as the number of adjacent biomarker nodes commonly connected to a node of the treatment method entity tⱼ and a node of the treatment method entity tₖ increases in the disease-specific bipartite graph of the disease entity dᵢ generated in step S403 in FIG. 4, a larger disease-specific inter-treatment-method similarity is calculated. When there is no adjacent biomarker node that is commonly connected, the disease-specific inter-treatment-method similarity is 0.

Step S703: the inter-node similarity calculation unit 109 calculates the disease-specific inter-biomarker similarity matrix B⁽ⁱ⁾ using the disease-specific adjacency matrix G⁽ⁱ⁾ for each disease entity dᵢ in the disease set 203. The disease-specific inter-biomarker similarity matrix B⁽ⁱ⁾ is an M-row M-column matrix whose rows and columns both correspond to the biomarker entities in the biomarker set 202 shown in FIG. 2, and a value B⁽ⁱ⁾ⱼₖ of a component in the j-th row and the k-th column represents a disease-specific inter-biomarker similarity between biomarker entities bⱼ and bₖ for the disease entity dᵢ (see FIG. 9).

$${B}_{j k}^{( i )} = \frac{1}{\sqrt{{f}_{2}^{( i )} ( {b}_{j} ) \cdot {f}_{2}^{( i )} ( {b}_{k} )}} {\sum }_{l = 1}^{L} \frac{{G}_{lj}^{( i )} \cdot {G}_{lk}^{( i )}}{{f}_{1}^{( i )} ( {t}_{l} )} \qquad Math 2$$

$${f}_{1}^{( i )} ( {t}_{j} ) := max \{ {\sum }_{k = 1}^{M} {G}_{j k}^{( i )} , 1 \}$$

$${f}_{2}^{( i )} ( {b}_{k} ) := max \{ {\sum }_{j = 1}^{L} {G}_{j k}^{( i )} , 1 \}$$

Formula 2 is a formula for calculating the disease-specific inter-biomarker similarity B⁽ⁱ⁾ⱼₖ between the biomarker entities bⱼ and bₖ for the disease entity dᵢ. In Formula 2, G⁽ⁱ⁾ⱼₖ, f⁽ⁱ⁾₁(tⱼ), and f⁽ⁱ⁾₂(bₖ) have the same values as G⁽ⁱ⁾ⱼₖ, f⁽ⁱ⁾₁(tⱼ), and f⁽¹⁾₂(bₖ) in Formula 1, respectively. In Formula 2, as the number of adjacent treatment method nodes commonly connected to a node of the biomarker entity bⱼ and a node of the biomarker entity bₖ increases in the disease-specific bipartite graph for the disease entity dᵢ generated in step S403 in FIG. 4, a larger disease-specific inter-biomarker similarity is calculated. When there is no adjacent treatment method node that is commonly connected, the disease-specific inter-biomarker similarity is 0.

Step S704: the inter-node similarity calculation unit 109 integrates the disease-specific adjacency matrix {G⁽¹⁾, G⁽²⁾, . . . , G^{(N)}} and a disease-specific inter-treatment-method similarity matrix {T⁽¹⁾, T⁽²⁾, . . . , T^{(N)}} to calculate the cross-disease inter-treatment-method similarity matrix T′. The cross-disease inter-treatment-method similarity matrix T′ is an L-row L-column matrix whose rows and columns both correspond to the treatment method entities in the treatment method set 201 shown in FIG. 2, and a value T′ⱼₖ of a component in the j-th row and the k-th column represents the cross-disease inter-treatment-method similarity between the treatment method entities tⱼ and tₖ. A data structure of the cross-disease inter-treatment-method similarity matrix T′ is the same as a data structure of the disease-specific inter-treatment-method similarity matrix T⁽ⁱ⁾ shown in FIG. 8, and has different component values.

$${T}_{jk}^{′} = \frac{1}{\sqrt{{g}_{1} ( {t}_{j} ) \cdot {g}_{1} ( {t}_{k} )}} {\sum }_{i = 1}^{N} {T}_{jk}^{( i )} \qquad Math 3$$

$${g}_{1} ( {t}_{j} ) := {\sum }_{i = 1}^{N} {h}_{1} ( {t}_{j} , {G}^{( i )} )$$

$${h}_{1} ( {t}_{j} , {G}^{( i )} ) := \{ \begin{matrix}1 & ( {\sum }_{k = 1}^{M} {G}_{jk}^{( i )} > 0 ) \\ 0 & ( OTHERWISE )\end{matrix}$$

Formula 3 is a formula for calculating the cross-disease inter-treatment-method similarity T′ⱼₖ between the treatment method entities tⱼ and tₖ. In Formula 3, G⁽ⁱ⁾ⱼₖ is the value of the component in the j-th row and the k-th column in the disease-specific adjacency matrix G⁽ⁱ⁾. A value of g₁(tⱼ) in Formula 3 is the number of disease-specific bipartite graphs in which the node degree of the treatment method entity tⱼ is larger than 0. A value of h₁(tⱼ, G⁽ⁱ⁾) in Formula 3 is 1 when the node degree of the treatment method entity tⱼ in the disease-specific bipartite graph G⁽ⁱ⁾ is larger than 0, and otherwise is 0. For the certain treatment method entities tⱼ and tₖ, when the value of the disease-specific inter-treatment-method similarity T⁽ⁱ⁾ⱼₖ is 0 for any disease entity dᵢ, the value of the cross-disease inter-treatment-method similarity T′ⱼₖ is 0.

Step S705: the inter-node similarity calculation unit 109 integrates the disease-specific adjacency matrix {G⁽¹⁾, G⁽²⁾, . . . , G^{(N)}} and a disease-specific inter-biomarker similarity matrix {B⁽¹⁾, B⁽²⁾, . . . , B^{(N)}} to calculate the cross-disease inter-biomarker similarity matrix B′. The cross-disease inter-biomarker similarity matrix B′ is an M-row M-column matrix whose rows and columns both correspond to the biomarker entities in the biomarker set 202 shown in FIG. 2, and a value B′ⱼₖ of a component in the j-th row and the k-th column represents a cross-disease inter-biomarker similarity between the biomarker entities bⱼ and bₖ. A data structure of the cross-disease inter-biomarker similarity matrix B′ is the same as a data structure of the disease-specific inter-biomarker similarity matrix B⁽ⁱ⁾ shown in FIG. 9, and has different component values.

$${B}_{jk}^{′} = \frac{1}{\sqrt{{g}_{2} ( {b}_{j} ) \cdot {g}_{2} ( {b}_{k} )}} {\sum }_{i = 1}^{N} {B}_{jk}^{( i )} \qquad Math 4$$

$${g}_{2} ( {b}_{k} ) := {\sum }_{i = 1}^{N} {h}_{2} ( {b}_{k} , {G}^{( i )} )$$

$${h}_{2} ( {b}_{k} , {G}^{( i )} ) := \{ \begin{matrix}1 & ( {\sum }_{j = 1}^{L} {G}_{jk}^{( i )} > 0 ) \\ 0 & ( OTHERWISE )\end{matrix}$$

Formula 4 is a formula for calculating the cross-disease inter-biomarker similarity B′ⱼₖ between the biomarker entities bⱼ and bₖ. In Formula 4, G⁽ⁱ⁾ⱼₖ is the value of the component in the j-th row and the k-th column in the disease-specific adjacency matrix G⁽ⁱ⁾. A value of g₂(bₖ) in Formula 4 is the number of disease-specific bipartite graphs in which the node degree of the biomarker entity bₖ is larger than 0. A value of h₂(bₖ, G⁽ⁱ⁾) in Formula 4 is 1 if the node degree of the biomarker entity bₖ in the disease-specific bipartite graph G⁽ⁱ⁾ is larger than 0, and otherwise is 0. For the certain biomarker entities bⱼ and bₖ, when the value of the disease-specific inter-biomarker similarity B⁽ⁱ⁾ⱼₖ is 0 for any disease entity dᵢ, the value of the cross-disease inter-biomarker similarity B′ⱼₖ is 0.

FIG. 8 shows the disease-specific inter-treatment-method similarity matrix T⁽ⁱ⁾ generated in step S702 in the inter-node similarity calculation processing shown in FIG. 7.

The disease-specific inter-treatment-method similarity matrix T⁽ⁱ⁾ is an L-row L-column matrix whose rows and columns both correspond to the treatment method entities in the treatment method set 201 shown in FIG. 2, and the value T⁽ⁱ⁾ⱼₖ of the component in the j-th row and the k-th column represents the disease-specific inter-treatment-method similarity between the treatment method entities tⱼ and tₖ for the disease entity dᵢ.

A component 801 in the first row and the first column in the disease-specific inter-treatment-method similarity matrix T⁽ⁱ⁾ indicates that the disease-specific inter-treatment-method similarity between treatment method entities t₁ and t₁ is 0.8 for the disease entity dᵢ.

FIG. 9 shows the disease-specific inter-biomarker similarity matrix B⁽ⁱ⁾ generated in step S703 in the inter-node similarity calculation processing shown in FIG. 7.

The disease-specific inter-biomarker similarity matrix B⁽ⁱ⁾ is an M-row M-column matrix whose rows and columns both correspond to the biomarker entities in the biomarker set 202 shown in FIG. 2, and the value B⁽ⁱ⁾ⱼₖ of the component in the j-th row and the k-th column represents a disease-specific inter-biomarker similarity between the biomarker entities bⱼ and bₖ for the disease entity dᵢ.

A component 901 in the first row and the first column in the disease-specific inter-biomarker similarity matrix B⁽ⁱ⁾ indicates that the disease-specific inter-biomarker similarity between biomarker entities b₁ and b₁ is 0.5 for the disease entity dᵢ.

FIG. 10 is a flowchart showing unknown edge prediction processing in the first embodiment of the invention.

The unknown edge prediction processing is executed by the unknown edge prediction unit 110 of the server 101.

Step S1001: the unknown edge prediction unit 110 acquires the disease set {d₁, d₂, . . . , d_{N}} 203, the disease-specific adjacency matrix {G⁽¹⁾, G⁽²⁾, . . . , G^{(N)}}, the disease-specific inter-treatment-method similarity matrix {T⁽¹⁾, T⁽²⁾, . . . , T^{(N)}}, the disease-specific inter-biomarker similarity matrix {B⁽¹⁾, B⁽²⁾, . . . , B^{(N)}}, the cross-disease inter-treatment-method similarity matrix T′, and the cross-disease inter-biomarker similarity matrix B′. The disease set 203 is acquired from the entity set data storage unit 112. The disease-specific adjacency matrix is generated in the disease-specific bipartite graph generation processing. The disease-specific inter-treatment-method similarity matrix, the disease-specific inter-biomarker similarity matrix, the cross-disease inter-treatment-method similarity matrix, and the cross-disease inter-biomarker similarity matrix are generated by the inter-node similarity calculation processing.

Step S1002: the unknown edge prediction unit 110 calculates, for each disease entity dᵢ in the disease set 203, the disease-specific prediction adjacency matrix P⁽ⁱ⁾ using the disease-specific adjacency matrix G⁽ⁱ⁾, the disease-specific inter-treatment-method similarity matrix T⁽ⁱ⁾, the cross-disease inter-treatment-method similarity matrix T′, the disease-specific inter-biomarker similarity matrix B⁽ⁱ⁾, and the cross-disease inter-biomarker similarity matrix B′. The disease-specific prediction adjacency matrix P⁽ⁱ⁾ is an L-row M-column matrix whose rows correspond to the treatment method entities in the treatment method set 201 shown in FIG. 2 and whose columns correspond to the biomarker entities in the biomarker set 202 shown in FIG. 2. A value P⁽ⁱ⁾ⱼₖ of a component in the j-th row and a k-th column in the disease-specific prediction adjacency matrix P⁽ⁱ⁾ represents a disease-specific prediction score of a binary relation between the treatment method entity tⱼ and the biomarker entity bₖ for the disease entity dᵢ. A higher value of the disease-specific prediction score P⁽ⁱ⁾ⱼₖ indicates a higher possibility that there is a binary relation between the treatment method entity tⱼ and the biomarker entity bₖ for the disease entity dᵢ. The disease-specific prediction score is calculated for any binary relation without distinguishing between a known binary relation and an unknown binary relation. A data structure of the disease-specific prediction adjacency matrix P⁽ⁱ⁾ is the same as the data structure of the disease-specific adjacency matrix G⁽ⁱ⁾ shown in FIG. 6, and has different component values.

$${P}^{( i )} = ( p {T}^{( i )} + {qT}^{′} ) {G}^{( i )} + {G}^{( i )} ( {uB}^{( i )} + ν {B}^{′} ) \qquad Math 5$$

$$p + q + u + v = 1$$

Formula 5 is a formula for calculating the disease-specific prediction adjacency matrix P⁽ⁱ⁾. In Formula 5, p, q, u, and v are hyperparameters having any value of 0 to 1, and determine a contribution rate of each matrix. Specifically, p is a disease-specific inter-treatment-method similarity weight for adjusting a contribution rate of the disease-specific inter-treatment-method similarity matrix T⁽ⁱ⁾. In addition, q is a cross-disease inter-treatment-method similarity weight for adjusting a contribution rate of the cross-disease inter-treatment-method similarity matrix T′. In addition, u is a disease-specific inter-biomarker similarity weight for adjusting a contribution rate of the disease-specific inter-biomarker similarity matrix B⁽ⁱ⁾. In addition, v is a cross-disease inter-biomarker similarity weight for adjusting a contribution rate of the cross-disease inter-biomarker similarity matrix B′.

When a sum p+u of the disease-specific inter-treatment-method similarity weight p and the disease-specific inter-biomarker similarity weight u is set to 1, only the disease-specific inter-treatment-method similarity matrix T⁽ⁱ⁾ and the disease-specific inter-biomarker similarity matrix B⁽ⁱ⁾ are used to calculate the disease-specific prediction adjacency matrix P⁽ⁱ⁾. At this time, since similarity information for any disease entity dᵢ different from the disease entity dᵢ, that is, edge information of the disease-specific bipartite graph for the disease entity dᵢ does not affect a calculated value of the disease-specific prediction adjacency matrix P⁽ⁱ⁾, a disease-specific prediction score of a false edge is low, indicating high prediction specificity. On the other hand, since the edge information of the disease-specific bipartite graph is not used for any disease entity dᵢ different from the disease entity dᵢ, the disease-specific prediction score is 0, that is, the number of edges that are practically impossible to predict increases, indicating low prediction sensitivity.

When a sum q+v of the cross-disease inter-treatment-method similarity weight q and the cross-disease inter-biomarker similarity weight v is set to 1, only the cross-disease inter-treatment-method similarity matrix T′ and the cross-disease inter-biomarker similarity matrix B′ are used to calculate the disease-specific prediction adjacency matrix P⁽ⁱ⁾. At this time, since the similarity information for any disease entity dᵢ different from the disease entity dᵢ, that is, the edge information of the disease-specific bipartite graph for the disease entity dᵢ affects the calculated value of the disease-specific prediction adjacency matrix P⁽ⁱ⁾, the number of edges whose disease-specific prediction score is larger than 0 increases, indicating high prediction sensitivity. The number of false edges also increases, indicating low prediction specificity.

That is, the sensitivity and the specificity of the prediction can be adjusted by adjusting the values of the hyperparameters.

Step S1003: the unknown edge prediction unit 110 calculates the cross-disease prediction adjacency matrix P′ using a disease-specific prediction adjacency matrix {P⁽¹⁾, P⁽²⁾, . . . , P^{(N)}}. The cross-disease prediction adjacency matrix P′ is an L-row M-column matrix whose rows correspond to the treatment method entities in the treatment method set 201 shown in FIG. 2 and whose columns correspond to the biomarker entities in the biomarker set 202 shown in FIG. 2. A value P′ⱼₖ of a component in the j-th row and the k-th column in the cross-disease prediction adjacency matrix P′ represents a cross-disease prediction score of the binary relation between the treatment method entity tⱼ and the biomarker entity bₖ. A higher value of the cross-disease prediction score P′ⱼₖ indicates a higher possibility that there is a binary relation between the treatment method entity tⱼ and the biomarker entity bₖ for a certain disease entity. The cross-disease prediction score is calculated for any binary relation without distinguishing between a known binary relation and an unknown binary relation. A data structure of the cross-disease prediction adjacency matrix P′ is the same as the data structure of the disease-specific adjacency matrix G⁽ⁱ⁾ shown in FIG. 6, and has different component values.

$${P}_{j k}^{′} = {max}_{i} {P}_{j k}^{( i )} \qquad Math 6$$

Formula 6 is a formula for calculating the cross-disease prediction score P′ⱼₖ of the binary relation between the treatment method entity tⱼ and the biomarker entity bₖ. In Formula 6, a maximum value of {P⁽¹⁾ⱼₖ, P⁽²⁾ⱼₖ, . . . , P^{(N)}ⱼₖ} is taken as the value of P′ⱼₖ.

FIG. 11 is a flowchart showing output processing in the first embodiment of the invention.

The output processing is mainly executed by the output unit 111 of the server 101.

Step S1101: the user inputs the values of the disease-specific inter-treatment-method similarity weight p, the cross-disease inter-treatment-method similarity weight q, the disease-specific inter-biomarker similarity weight u, and the cross-disease inter-biomarker similarity weight v to the input apparatus 103.

Step S1102: based on the values input by the user in step S1101, the disease-specific prediction adjacency matrix {P⁽¹⁾, P⁽²⁾, . . . , P^{(N)}} and the cross-disease prediction adjacency matrix P′ are calculated by the unknown edge prediction processing. The unknown edge prediction processing is executed by the unknown edge prediction unit 110 of the server 101.

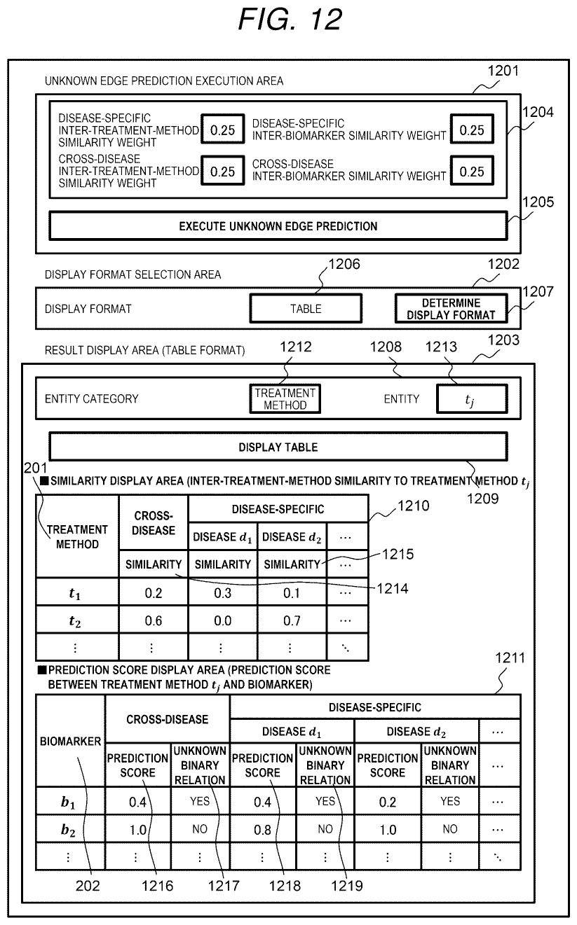

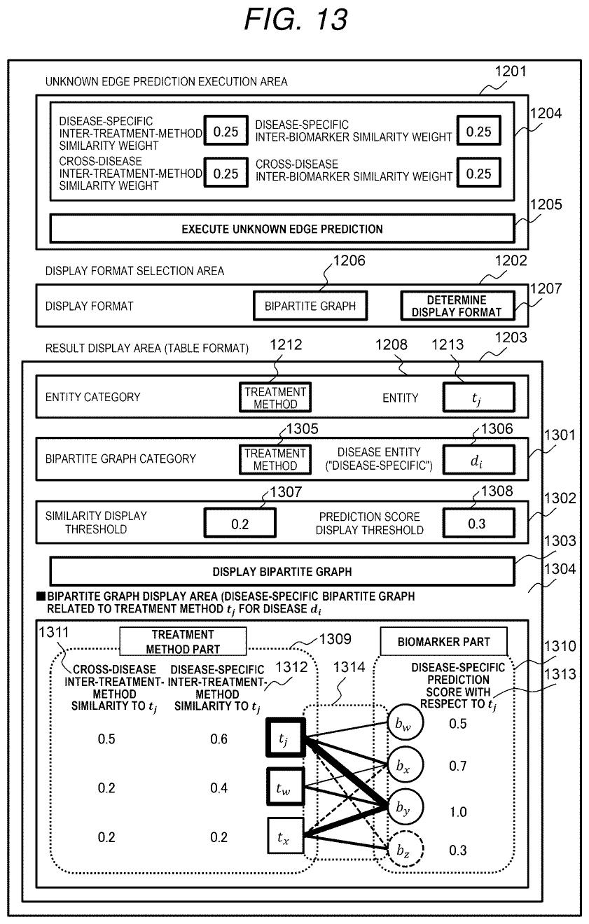

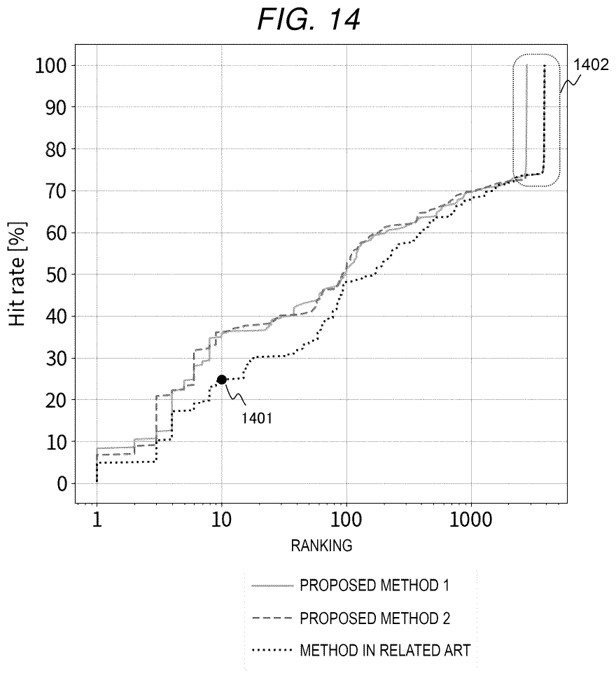

Step S1103: the user inputs a result display format to a display format input area 1206 (see FIGS. 12 and 13). An input value is “table format” or “bipartite graph format”. When the input value is “table format”, the processing proceeds to step S1104, and an operation screen shown in FIG. 12 is presented to the user. When the input value is “bipartite graph format”, the processing proceeds to step S1107, and an operation screen shown in FIG. 13 is presented to the user.

Step S1104: the user inputs a treatment method entity or biomarker entity that is a result display target to an entity input area 1213 (see FIG. 12). An input value is any element in the treatment method set {t₁, t₂, . . . , t_{L}} 201 or any element in the biomarker set 202 {b₁, b₂, . . . , b_{M}}.

Step S1105: data to be displayed is extracted based on the value input by the user in step S1104.

For example, when the input value is the treatment method entity tⱼ, the j-th row in the cross-disease inter-treatment-method similarity matrix T′, the j-th row in the disease-specific inter-treatment-method similarity matrix T⁽ⁱ⁾ of each disease entity dᵢ, the j-th row in the cross-disease prediction adjacency matrix P′, the j-th row in the disease-specific prediction adjacency matrix P⁽ⁱ⁾ of each disease entity dᵢ, the j-th row in the cross-disease adjacency matrix G′, and the j-th row in the disease-specific adjacency matrix G⁽ⁱ⁾ of each disease entity dᵢ are extracted.

For example, when the input value is the biomarker entity bₖ, the k-th column in the cross-disease inter-biomarker similarity matrix B′, the k-th column in the disease-specific inter-biomarker similarity matrix B⁽ⁱ⁾ of each disease entity dᵢ, the k-th column in the cross-disease prediction adjacency matrix P′, the k-th column in the disease-specific prediction adjacency matrix P⁽ⁱ⁾ of each disease entity dᵢ, the k-th column in the cross-disease adjacency matrix G′, and the k-th column in the disease-specific adjacency matrix G⁽ⁱ⁾ for each disease entity dᵢ are extracted.

Step S1106: the output unit 111 displays the data extracted in step S1105 in the table format (see FIG. 12).

Step S1107: when the input value is “bipartite graph format”, the user inputs the treatment method entity or biomarker entity that is the result display target to the entity input area 1213 (see FIG. 13). An input value is any element in the treatment method set {t₁, t₂, . . . , t_{L}} 201 or any element in the biomarker set 202 {b₁, b₂, . . . , b_{M}}.

Step S1108: the user inputs a bipartite graph category. The bipartite graph category is “disease-specific” or “cross-disease”. When the input value is “disease-specific”, the processing proceeds to step S1109, and the disease-specific bipartite graph is displayed. When the input value is “cross-disease”, the processing proceeds to step S1110, and the cross-disease bipartite graph is displayed.

Step S1109: the user inputs the disease entity whose result is to be displayed. An input value is any element in the disease set {d₁, d₂, . . . , d_{N}} 203. When the input value is dᵢ, a displayed bipartite graph is the disease-specific bipartite graph for the disease entity dᵢ.

Step S1110: the user inputs a similarity display threshold and a prediction score display threshold.

Step S1111: the output unit 111 extracts data for displaying the result in the bipartite graph format based on the input values of the user from step S1107 to step S1110.

For example, a case where the input value in step S1107 is tⱼ, the input value in step S1108 is “disease-specific”, and the input value in step S1109 is dᵢ will be described. All treatment method entities whose cross-disease inter-treatment-method similarity to the input value tⱼ is equal to or higher than the similarity display threshold, or whose disease-specific inter-treatment-method similarity to the input value tⱼ for the disease entity dᵢ is equal to or higher than the similarity display threshold are extracted as treatment method nodes to be displayed in the bipartite graph. At this time, the inter-treatment-method similarity is extracted in order to be reflected in treatment method node border thickness. Next, all biomarker entities whose disease-specific prediction score with respect to the input value tⱼ for the disease entity dᵢ is equal to or higher than the prediction score display threshold are extracted as biomarker nodes to be displayed in the bipartite graph. At this time, the prediction score is extracted in order to be reflected in bipartite graph edge thickness. Next, whether there is any known binary relation between the extracted treatment method entity and the extracted biomarker entity is extracted from the disease-specific adjacency matrix G⁽ⁱ⁾ of the disease entity dᵢ in order to be reflected in edge line types and biomarker node line types of the bipartite graph. For example, an edge corresponding to a known binary relation may be displayed by a solid line, and an edge corresponding to an unknown binary relation may be displayed by a broken line. A border of a biomarker node having a known binary relation to the input value tⱼ is displayed by a solid line, and a border of a biomarker node having no known binary relation is displayed by a broken line.

For example, a case where the input value in step S1107 is tⱼ, and an input value in step S1108 is “cross-disease” will be described. All treatment method entities whose cross-disease inter-treatment-method similarity to the input value tⱼ is equal to or higher than the similarity display threshold are extracted as the treatment method nodes to be displayed in the bipartite graph. The inter-treatment-method similarity is extracted in order to be reflected in the treatment method node border thickness. Next, all biomarker entities whose cross-disease prediction score with respect to the input value tⱼ is equal to or higher than the prediction score display threshold are extracted as the biomarker nodes to be displayed in the bipartite graph. The prediction score is extracted in order to be reflected in the bipartite graph edge thickness. Next, whether there is any known binary relation between the extracted treatment method entity and the extracted biomarker entity is extracted from the cross-disease adjacency matrix G′ in order to be reflected in the edge line types and the biomarker node line types of the bipartite graph. For example, an edge corresponding to a known binary relation may be displayed by a solid line, and an edge corresponding to an unknown binary relation may be displayed by a broken line. A border of a biomarker node having a known binary relation to the input value tⱼ is displayed by a solid line, and a border of a biomarker node having no known binary relation is displayed by a broken line.

For example, a case where the input value in step S1107 is bₖ, the input value in step S1108 is “disease-specific”, and the input value in step S1109 is dᵢ will be described. All biomarker entities whose cross-disease inter-biomarker similarity to the input value bₖ is equal to or higher than the similarity display threshold, or whose disease-specific inter-biomarker similarity to the input value bₖ for the disease entity dᵢ is equal to or higher than the similarity display threshold are extracted as biomarker nodes to be displayed in the bipartite graph. The inter-biomarker similarity is extracted in order to be reflected in biomarker node border thickness. Next, all treatment method entities whose disease-specific prediction score for the disease entity dᵢ with respect to the input value bₖ is equal to or higher than the prediction score display threshold are extracted as the treatment method nodes to be displayed in the bipartite graph. The prediction score is extracted in order to be reflected in the bipartite graph edge thickness. Next, whether there is any known binary relation between the extracted treatment method entity and the extracted biomarker entity is extracted from the disease-specific adjacency matrix G⁽ⁱ⁾ of the disease entity dᵢ in order to be reflected in the edge line types and the biomarker node line types of the bipartite graph. For example, an edge corresponding to a known binary relation may be displayed by a solid line, and an edge corresponding to an unknown binary relation may be displayed by a broken line. A border of a treatment method node having a known binary relation to the input value bₖ may be displayed by a solid line, and a border of a treatment method node having no known binary relation may be displayed by a broken line.

For example, a case where the input value in step S1107 is bₖ, and the input value in step S1108 is “cross-disease” will be described. All biomarker entities whose cross-disease inter-biomarker similarity to the input value bₖ is equal to or higher than the similarity display threshold are extracted as the biomarker nodes to be displayed in the bipartite graph. The inter-biomarker similarity is extracted in order to be reflected in the biomarker node border thickness. Next, all treatment method entities whose cross-disease prediction score with respect to the input value bₖ is equal to or higher than the prediction score display threshold are extracted as the treatment method nodes to be displayed in the bipartite graph. The prediction score is extracted in order to be reflected in the bipartite graph edge thickness. Next, whether there is any known binary relation between the extracted treatment method entity and the extracted biomarker entity is extracted from the cross-disease adjacency matrix G′ in order to be reflected in the edge line types and the biomarker node line types of the bipartite graph. For example, an edge corresponding to a known binary relation may be displayed by a solid line, and an edge corresponding to an unknown binary relation may be displayed by a broken line. A border of a treatment method node having a known binary relation to the input value bₖ may be displayed by a solid line, and a border of a treatment method node having no known binary relation may be displayed by a broken line.

Step S1112: the output unit 111 displays the data extracted in step S1111 in the bipartite graph format (see FIG. 13).

FIG. 12 shows an example of an operation screen for displaying a result output in the output processing in the first embodiment of the invention in a table format.

The user can refer to a calculation result comprehensively through the operation screen shown in FIG. 12. For example, a drug researcher can use when searching for an unknown biomarker for a treatment method of interest. However, a use method is not limited to this example.

The operation screen shown in FIG. 12 includes an unknown edge prediction execution area 1201, a display format selection area 1202, and a result display area 1203.

The unknown edge prediction execution 1201 includes a weight input area 1204 and an unknown edge prediction execution button 1205.

First, the user inputs the value of the disease-specific inter-treatment-method similarity weight p, the value of the cross-disease inter-treatment-method similarity weight q, the value of the disease-specific inter-biomarker similarity weight u, and the value of the cross-disease inter-biomarker similarity weight v to the weight input area 1204. Only a value of 0 or more can be input as a weight value.

Next, according to an operation on the unknown edge prediction execution button 1205 by the user, the unknown edge prediction unit 110 of the server 101 executes the unknown edge prediction processing based on the values of the weights p, q, u, and v input to the weight input area 1204. At this time, the values of the weights p, q, u, and v input to the weight input area 1204 are normalized to p/(p+q+u+v), q/(p+q+u+v), u/(p+q+u+v), and v/(p+q+u+v) such that a sum p+q+u+v is 1. When the values of the weights p, q, u, and v input to the weight input area 1204 are all 0, the unknown edge prediction processing may not be executed, and a prompt may be displayed to prompt the user to set one or more weight values of p, q, u, and v to be positive.

The display format selection area 1202 includes the display format input area 1206 and a display format determination button 1207. The display format selection area 1202 is displayed after the unknown edge prediction execution button 1205 is operated and the unknown edge prediction processing is executed.

First, the user inputs a display format to the display format input area 1206. A value of the input display format is “table” or “bipartite graph”. When “table” is input to the display format input area 1206, a result is displayed in the result display area 1203 in a table format. When “bipartite graph” is input to the display format input area 1206, the result is displayed in the result display area 1203 in a bipartite graph format (see FIG. 13). Next, the result is displayed in the result display area 1203 by operating the display format determination button 1207. FIG. 12 shows a case where “table” is input to the display format input area 1206.

In the result display area 1203 in FIG. 12, the result in the table format is displayed. When the result in the table format is displayed, the result display area 1203 includes an entity selection area 1208, a table display button 1209, a similarity display area 1210, and a prediction score display area 1211. The similarity display area 1210 and the prediction score display area 1211 are displayed after the table display button 1209 is operated.

The entity selection area 1208 includes an entity category input area 1212 and the entity input area 1213.

First, the user inputs an entity category into the entity category input area 1212. A value of the entity category is “treatment method” or “biomarker”. Next, the user inputs an entity into the entity input area 1213 and inputs a specific treatment method or biomarker to be displayed. When the entity category is “treatment method”, the entity is any element in the treatment method set {t₁, t₂, . . . , t_{L}} 201. When the entity category is “biomarker”, the entity is any element in the biomarker set 202 {b₁, b₂, . . . , b_{M}}. FIG. 12 shows a case where “treatment method” is input as the entity category and tⱼ is input as the entity.

Next, the user operates the table display button 1209. Accordingly, a result related to the entity input to the entity selection area 1208 is displayed in the similarity display area 1210 and the prediction score display area 1211.

The similarity display area 1210 displays, for each treatment method entity tₖ in the treatment method set {t₁, t₂, . . . , t_{L}} 201 and each disease entity dᵢ in the disease set {d₁, d₂, . . . , d_{M}} 203, a cross-disease inter-treatment-method similarity 1214 between the treatment method entities tⱼ and tₖ and a disease-specific inter-treatment-method similarity 1215 between the treatment method entities tⱼ and tₖ for the disease entity dᵢ. When “biomarker” is assumed to be input to the entity category input area 1212, the similarity display area 1210 displays, for each biomarker entity bₖ in the biomarker set {b₁, b₂, . . . , b_{M}} 202 and each disease entity dᵢ in the disease set {d₁, d₂, . . . , d_{M}} 203, the cross-disease inter-biomarker similarity between the biomarker entities bⱼ and bₖ and the disease-specific inter-biomarker similarity between the biomarker entities bⱼ and bₖ for the disease entity dᵢ. Data in the similarity display area 1210 can be sorted according to values of any one or more columns.

A value of the cross-disease inter-treatment-method similarity 1214 between the treatment method entities tⱼ and tₖ is the component value T′ⱼₖ in the j-th row and the k-th column in the cross-disease inter-treatment-method similarity matrix T′.

A value of the disease-specific inter-treatment-method similarity 1215 between the treatment method entities tⱼ and tₖ for the disease entity dᵢ is the component value T⁽ⁱ⁾ⱼₖ in the j-th row and the k-th column in the disease-specific inter-treatment-method similarity matrix T⁽ⁱ⁾.

The prediction score display area 1211 displays, for each biomarker entity bₖ in the biomarker set {b₁, b₂, . . . , b_{M}} 202, a cross-disease prediction score 1216 between the treatment method entity tⱼ and the biomarker entity bₖ, whether a binary relation between the treatment method entity tⱼ and the biomarker entity bₖ for all disease entities in the disease set {d₁, d₂, . . . , d_{L}} 203 is unknown 1217, a disease-specific prediction score 1218 between the treatment method entity tⱼ and the biomarker entity bₖ for each disease entity dᵢ in the disease set {d₁, d₂, . . . , d_{L}} 203, and whether the binary relation between the treatment method entity tⱼ and the biomarker entity bₖ for each disease entity dᵢ in the disease set {d₁, d₂, . . . , d_{L}} 203 is unknown 1219. When “biomarker” is assumed to be input to the entity category input area 1212, the prediction score display area 1211 displays, for each treatment method entity t; in the treatment method set {t₁, t₂, . . . , t_{L}} 201, the cross-disease prediction score between the biomarker entity bₖ and the treatment method entity tⱼ, whether the binary relation between the biomarker entity bₖ and the treatment method entity tⱼ for all disease entities in the disease set {d₁, d₂, . . . , d_{L}} 203 is unknown, the disease-specific prediction score between the biomarker entity bₖ and the treatment method entity tⱼ for each disease entity dᵢ in the disease set {d₁, d₂, . . . , d_{L}} 203, and whether the binary relation between the biomarker entity bₖ and the treatment method entity tⱼ for each disease entity dᵢ in the disease set {d₁, d₂, . . . , d_{L}} 203 is unknown. Data in the prediction score display area 1211 can be sorted according to values of any one or more columns.

A value of the cross-disease prediction score 1216 between the treatment method entity tⱼ and the biomarker entity bₖ is the component value P′ⱼₖ in the j-th row and the k-th column in the cross-disease prediction adjacency matrix P′.

A value of whether the binary relation between the treatment method entity tⱼ and the biomarker entity bₖ for all the disease entities dᵢ in the disease set 203 is unknown 1217 is “YES” when a value G′ⱼₖ in the j-th row and the k-th column in the cross-disease adjacency matrix is 0, and otherwise is “NO”.

A value of the disease-specific prediction score 1218 between the treatment method entity tⱼ and the biomarker entity bₖ for the disease entity dᵢ is the component value P⁽ⁱ⁾ⱼₖ in the j-th row and the k-th column in the disease-specific prediction adjacency matrix P⁽ⁱ⁾.

A value of whether the binary relation between the treatment method entity tⱼ and the biomarker entity bₖ for the disease entity dᵢ is unknown 1219 is “YES” when the value G⁽ⁱ⁾ⱼₖ in the j-th row and the k-th column in the disease-specific adjacency matrix of the disease entity dᵢ is 0, and otherwise is “NO”.

In the example of the operation screen shown in FIG. 12, all of the disease-specific inter-treatment-method similarity, the cross-disease inter-treatment-method similarity, the disease-specific inter-biomarker similarity, the cross-disease inter-biomarker similarity, the disease-specific prediction score, and the cross-disease prediction score are displayed, and alternatively, any one thereof may be displayed depending on an application. In the example of the operation screen shown in FIG. 12, both the treatment method and the biomarker are displayed, and alternatively, either the treatment method or the biomarker may be displayed depending on the application.

FIG. 13 shows an example of an operation screen for displaying the result output in the output processing in the first embodiment of the invention in a bipartite graph format.

The user can easily visually recognize known and unknown binary relations through the operation screen shown in FIG. 13 using a bipartite graph, and can adjust a node and an edge displayed in the bipartite graph by threshold processing.

The operation screen shown in FIG. 13 includes the unknown edge prediction execution area 1201, the display format selection area 1202, and the result display area 1203. FIG. 13 shows a case where “bipartite graph” is input to the display format input area 1206 and the result is displayed in the bipartite graph format in the result display area 1203.

Since the unknown edge prediction execution area 1201 and the display format selection area 1202 are the same as those in FIG. 12, description thereof will be omitted.

In the result display area 1203 in FIG. 13, the result in the bipartite graph format is displayed. When the result in the bipartite graph format is displayed, the result display area 1203 includes the entity selection area 1208, a bipartite graph selection area 1301, a threshold setting area 1302, a bipartite graph display button 1303, and a bipartite graph display area 1304. The bipartite graph display area 1304 is displayed after the bipartite graph display button 1303 is operated.

Since the entity selection area 1208 is the same as that in FIG. 12, description thereof will be omitted. FIG. 13 shows a case where “treatment method” is input as the entity category and tⱼ is input as the entity.

The bipartite graph selection area 1301 includes a bipartite graph category input area 1305 and a disease entity input area 1306.

The user inputs a bipartite graph category to the bipartite graph category input area 1305. A value of the bipartite graph category is “disease-specific” or “cross-disease”. When the bipartite graph category is “disease-specific”, next, the user inputs the disease entity to the disease entity input area 1306. The disease entity is any element in the disease set {d₁, d₂, . . . , d_{N}} 203. FIG. 13 shows a case where “disease-specific” is input as the bipartite graph category and dᵢ is input as the disease entity.

The threshold setting area 1302 includes a similarity display threshold input area 1307 and a prediction score display threshold input area 1308. FIG. 13 shows a case where 0.2 is input to the similarity display threshold input area 1307 and 0.3 is input to the prediction score display threshold input area 1308.

After inputting to the entity selection area 1208, the bipartite graph selection area 1301, and the threshold setting area 1302, the user can display a bipartite graph in the bipartite graph display area 1304 by operating the bipartite graph display button 1303.

In the bipartite graph display area 1304, since the treatment method entity tⱼ is input in the entity selection area 1208, “disease-specific” is input as the bipartite graph category, and dᵢ is input as the disease entity in the bipartite graph selection area 1301, a disease-specific bipartite graph related to the treatment method entity tⱼ for the disease entity dᵢ is displayed. When “cross-disease” is input as the bipartite graph category, the bipartite graph display area 1304 displays a cross-disease bipartite graph related to the treatment method entity tⱼ.

The treatment method part 1309 displays a treatment method entity node whose cross-disease inter-treatment-method similarity 1311 to the treatment method entity tⱼ or whose disease-specific inter-treatment-method similarity 1312 to the treatment method entity tⱼ for the disease entity dᵢ is equal to or higher than 0.2, which is the value input to the similarity display threshold input area 1307. Next to each treatment method node, the cross-disease inter-treatment-method similarity 1311 to the treatment method entity tⱼ and the disease-specific inter-treatment-method similarity 1312 to the treatment method entity tⱼ for the disease entity dᵢ are displayed. A border of each treatment method node may be drawn thicker as an average value of the cross-disease inter-treatment-method similarity 1311 and the disease-specific inter-treatment-method similarity 1312 increases.

In a biomarker part 1310, a biomarker entity node whose disease-specific prediction score 1313 with respect to the treatment method entity tⱼ for the disease entity dᵢ is equal to or higher than 0.3, which is the value input to the prediction score display threshold input area 1308, is displayed. Next to each biomarker node, the disease-specific prediction score 1313 with respect to the treatment method entity tⱼ is displayed. A border of each biomarker node may be displayed by a solid line when there is a known binary relation to the treatment method entity tⱼ, and may be displayed by a broken line when there is no known binary relation.

A bipartite graph edge 1314 may be drawn thicker as the disease-specific prediction score for the disease entity dᵢ between the treatment method entity and the biomarker entity to which the edge is connected increases. The edge may be displayed by a solid line when there is a known binary relation between the treatment method entity and the biomarker entity to which the edge is connected, and may be displayed by a broken line when there is no known binary relation.

In the example of the operation screen shown in FIG. 13, all of the disease-specific inter-treatment-method similarity, the cross-disease inter-treatment-method similarity, the disease-specific inter-biomarker similarity, the cross-disease inter-biomarker similarity, the disease-specific prediction score, and the cross-disease prediction score are displayed, and alternatively, any one thereof may be displayed depending on an application. In the example of the operation screen shown in FIG. 13, both the treatment method and the biomarker are displayed, and alternatively, either the treatment method or the biomarker may be displayed depending on the application.

As described above, the information processing system in the first embodiment can predict the unknown relation between the treatment method and the biomarker with high accuracy by generating the disease-specific bipartite graph of the treatment method and the biomarker, calculating the disease-specific inter-node similarity and the cross-disease inter-node similarity based on the generated disease-specific bipartite graph, and performing edge prediction using the calculated inter-node similarities.

The prediction of the disease-specific binary relation between the treatment method and the biomarker substantially corresponds to prediction of the ternary relation among the treatment method, the biomarker, and the disease. That is, it is possible to predict the binary relation between the treatment method and the biomarker using the cross-disease prediction adjacency matrix, and to predict the ternary relation among the treatment method, the biomarker, and the disease using the disease-specific prediction adjacency matrix. Although the first embodiment has been described in relation to the prediction of the unknown relation between the treatment method and the biomarker, the information processing system in the first embodiment can receive biomarker information or treatment method information of a patient as an input at the time of examination by a doctor, and then can be used for supporting selection of a treatment method suitable for a symptom of the patient based on an unknown relation predicted according to the first embodiment.

### Second Embodiment

In the present embodiment, edge prediction specificity is lowered, and edge prediction sensitivity is increased. In a second embodiment, only a configuration and processing different from those in the first embodiment are described with reference to FIGS. 1 to 13, and description of the same configuration and processing as those in the first embodiment is omitted.

In the inter-node similarity calculation processing shown in FIG. 7, a calculation method for the cross-disease inter-treatment-method similarity matrix in step S704 and a calculation method for the cross-disease inter-biomarker similarity matrix in step S705 are different from those in the first embodiment.

Step S704: the inter-node similarity calculation unit 109 calculates the cross-disease inter-treatment-method similarity matrix T′ using the cross-disease adjacency matrix G′. The cross-disease inter-treatment-method similarity matrix T′ is an L-row L-column matrix whose rows and columns both correspond to the treatment method entities in the treatment method set 201 shown in FIG. 2, and the value T′ⱼₖ of the component in the j-th row and the k-th column represents the cross-disease inter-treatment-method similarity between the treatment method entities tⱼ and tₖ. The data structure of the cross-disease inter-treatment-method similarity matrix T′ is the same as the data structure of the disease-specific inter-treatment-method similarity matrix T⁽ⁱ⁾ shown in FIG. 8, and has different component values.

$${T}_{j k}^{′} = \frac{1}{\sqrt{{f}_{1}^{′} ( {t}_{j} ) \cdot {f}_{1}^{′} ( {t}_{k} )}} {\sum }_{l = 1}^{M} \frac{{G}_{j l}^{′} \cdot {G}_{k l}^{′}}{{f}_{2}^{′} ( {b}_{l} )} \qquad Math 7$$

$${f}_{1}^{′} ( {t}_{j} ) := max \{ {\sum }_{k = 1}^{M} {G}_{j k}^{′} , 1 \}$$

$${f}_{2}^{′} ( {b}_{k} ) := max \{ {\sum }_{j = 1}^{L} {G}_{j k}^{′} , 1 \}$$

Formula 7 is a formula for calculating the cross-disease inter-treatment-method similarity T′ⱼₖ between the treatment method entities tⱼ and tₖ. The value of G′ⱼₖ in Formula 7 is the value of the component in the j-th row and the k-th column in the cross-disease adjacency matrix G′. A value of f′₁(tⱼ) in Formula 7 is the node degree of the treatment method entity tⱼ in the cross-disease bipartite graph. A value of f′₂(bₖ) in Formula 7 is the node degree of the biomarker entity bₖ in the cross-disease bipartite graph. In Formula 7, as the number of adjacent biomarker nodes commonly connected to the node of the treatment method entity tⱼ and the node of the treatment method entity tₖ increases in the cross-disease bipartite graph generated in step S407 in FIG. 4, a larger similarity is calculated. When there is no adjacent biomarker node that is commonly connected, the cross-disease inter-treatment-method similarity is 0.

Step S705: the inter-node similarity calculation unit 109 calculates the cross-disease inter-biomarker similarity matrix B′ using the cross-disease adjacency matrix G′. The cross-disease inter-biomarker similarity matrix B′ is an M-row M-column matrix whose rows and columns both correspond to the biomarker entities in the biomarker set 202 shown in FIG. 2, and the value B′ⱼₖ of the component in the j-th row and the k-th column represents the cross-disease inter-biomarker similarity between the biomarker entities bⱼ and bₖ. The data structure of the cross-disease inter-biomarker similarity matrix B′ is the same as the data structure of the disease-specific inter-biomarker similarity matrix B⁽ⁱ⁾ shown in FIG. 9, and has different component values.

$${B}_{j k}^{′} = \frac{1}{\sqrt{{f}_{2}^{′} ( {b}_{j} ) \cdot {f}_{2}^{′} ( {b}_{k} )}} {\sum }_{l = 1}^{L} \frac{{G}_{lj}^{′} \cdot {G}_{lk}^{′}}{{f}_{1}^{′} ( {t}_{l} )} \qquad Math 8$$

$${f}_{1}^{′} ( {t}_{j} ) := max \{ {\sum }_{k = 1}^{M} {G}_{j k}^{′} , 1 \}$$

$${f}_{2}^{′} ( {b}_{k} ) := max \{ {\sum }_{j = 1}^{L} {G}_{j k}^{′} , 1 \}$$

Formula 8 is a formula for calculating the cross-disease inter-biomarker similarity B′ⱼₖ between the biomarker entities bⱼ and bₖ. Here, G′ⱼₖ, f′₁(tⱼ), and f′₂(bₖ) in Formula 8 are the same as G′ⱼₖ, f′₁(tⱼ), and f′₂(bₖ) in Formula 7, respectively. In Formula 8, as the number of adjacent treatment method nodes commonly connected to the node of the biomarker entity bⱼ and the node of the biomarker entity bₖ increases in the cross-disease bipartite graph generated in step S407 in FIG. 4, a larger similarity is calculated. When there is no adjacent treatment method node that is commonly connected, the cross-disease inter-biomarker similarity is 0.

Regarding step S704, in the first embodiment, the cross-disease inter-treatment-method similarity matrix T′ is calculated using Formula 3 based on the disease-specific adjacency matrix {G⁽¹⁾, G⁽²⁾, . . . , G^{(N)}} and the disease-specific inter-treatment-method similarity matrix {T⁽¹⁾, T⁽²⁾, . . . , T^{(N)}}. In the first embodiment, when the value of T⁽ⁱ⁾ⱼₖ for any disease entity dᵢ is 0 for certain treatment method entities tⱼ and tₖ, the value of T′ⱼₖ is 0. On the other hand, in the second embodiment, the cross-disease inter-treatment-method similarity matrix T′ is calculated using Formula 7 based on the cross-disease adjacency matrix G′. In the second embodiment, since the cross-disease adjacency matrix generated ignoring disease information is used, even when the value of T⁽ⁱ⁾ⱼₖ for any disease entity dᵢ is 0 for certain treatment method entities tⱼ and tₖ, the value of T′ⱼₖ may be higher than 0. Therefore, the number of elements whose cross-disease inter-treatment-method similarity matrix T′ calculated in the second embodiment is not 0 is equal to or more than the number of elements whose cross-disease inter-treatment-method similarity matrix T′ calculated in the first embodiment is not 0. Since the disease-specific prediction adjacency matrix P⁽ⁱ⁾ is calculated using Formula 5, it is expected that the number of elements whose disease-specific prediction adjacency matrix P⁽ⁱ⁾ is not 0 increases as the number of elements whose cross-disease inter-treatment-method similarity matrix T′ is not 0 increases. This may lead to a decrease in edge prediction specificity and may also lead to an increase in sensitivity.

Regarding step S705, in the first embodiment, the cross-disease inter-biomarker similarity matrix B′ is calculated using Formula 4 based on the disease-specific adjacency matrix {G⁽¹⁾, G⁽²⁾, . . . , G^{(N)}} and the disease-specific inter-biomarker similarity matrix {B⁽¹⁾, B⁽²⁾, . . . , B^{(N)}}. In the first embodiment, when the value of B⁽ⁱ⁾ⱼₖ for any disease entity dᵢ is 0 for certain biomarker entities bⱼ and bₖ, the value of B′ⱼₖ is 0. On the other hand, in the second embodiment, the cross-disease inter-biomarker similarity matrix B′ is calculated using Formula 8 based on the cross-disease adjacency matrix G′. In the second embodiment, since the cross-disease adjacency matrix generated ignoring disease information is used, even when the value of B⁽ⁱ⁾ⱼₖ for any disease entity dᵢ is 0 for certain biomarker entities bⱼ and bₖ, the value of B′ⱼₖ may be higher than 0. Therefore, the number of elements whose cross-disease inter-biomarker similarity matrix B′ calculated in the second embodiment is not 0 is equal to or more than the number of elements whose cross-disease inter-biomarker similarity matrix B′ calculated in the first embodiment is not 0. Since the disease-specific prediction adjacency matrix P⁽ⁱ⁾ is calculated using Formula 5, it is expected that the number of elements whose disease-specific prediction adjacency matrix P⁽ⁱ⁾ is not 0 increases as the number of elements whose cross-disease inter-biomarker similarity matrix B′ is not 0 increases. This may lead to a decrease in edge prediction specificity and may also lead to an increase in sensitivity.

As described above, the information processing system in the second embodiment can lower relation prediction specificity and increase relation prediction sensitivity by changing the calculation method for the cross-disease inter-treatment-method similarity matrix in step S704 in the first embodiment and the calculation method for the cross-disease inter-biomarker similarity matrix in step S705 in the first embodiment.

The unknown binary relation between the treatment method and the biomarker predicted by the information processing systems in the first and second embodiments described above can be used for supporting treatment method selection based on a biomarker detected in a patient in clinical practice. The predicted unknown binary relation between the treatment method and the biomarker can be used for predicting a degree of treatment efficacy by being input to a treatment efficacy prediction system different from the invention.

Next, effects of the information processing systems in the first and second embodiments will be described in comparison with an information processing system in related art that does not consider disease information. In the following description, the information processing system in the first embodiment is referred to as a “proposed method 1”, the information processing system in the second embodiment is referred to as a “proposed method 2”, and the information processing system in the related art that does not consider the disease is referred to as a “method in related art”.

In the method in related art, the cross-disease adjacency matrix G′, the cross-disease inter-treatment-method similarity matrix T′, and the cross-disease inter-biomarker similarity matrix B′ are calculated by the same procedure as in the proposed method 2. Meanwhile, in the method in related art, the disease-specific adjacency matrix {G⁽¹⁾, G⁽²⁾, . . . . G^{(N)}}, the disease-specific inter-treatment-method similarity matrix {T⁽¹⁾, T⁽²⁾, . . . , T^{(N)}}, the disease-specific inter-biomarker similarity matrix {B⁽¹⁾, B⁽²⁾, . . . , B^{(N)}}, and the disease-specific prediction adjacency matrix {P⁽¹⁾, P⁽²⁾, . . . , P^{(N)}} are not calculated. A calculation method for the cross-disease prediction adjacency matrix P′ in the method in related art is different from the calculation methods for the cross-disease prediction adjacency matrix P′ in the proposed method 1 and the proposed method 2. Therefore, the calculation method for the cross-disease prediction adjacency matrix P′ in the method in related art will be described first.

$${P}^{′} = {qT}^{′} {G}^{′} + {vG}^{′} {B}^{′} \qquad Math 9$$

$$q + v = 1$$

Formula 9 is a formula for calculating the cross-disease prediction adjacency matrix P′ in the method in related art. Here, q and v are hyperparameters having any value of 0 to 1. In addition, q is the cross-disease inter-treatment-method similarity weight for adjusting the contribution rate of the cross-disease inter-treatment-method similarity matrix T′. In addition, v is the cross-disease inter-biomarker similarity weight for adjusting the contribution rate of the cross-disease inter-biomarker similarity matrix B′.

In order to compare prediction accuracy of the proposed method 1, the proposed method 2, and the method in related art, a leave-one-out cross-validation is performed for each method. The “leave-one-out cross-validation” is a verification method of repeating, iteratively for all samples in a data set, a process of removing one sample from a data set as a test sample and performing a prediction evaluation for the test sample using the remaining data.

As data for the leave-one-out cross-validation, data of 824 sets of known ternary relations confirmed in clinical and experimental manners in relation to the treatment method entity, the biomarker entity, and the disease entity in a public database TheMarker (https://themarker.idrblab.cn) is used. In each trial of the leave-one-out cross-validation in the present embodiment, a certain known ternary relation (tⱼ, bₖ, dₗ) among the treatment method entity tⱼ, the biomarker entity bₖ, and the disease entity dₗ is removed from the 824 sets of the ternary relation data 300, the cross-disease prediction adjacency matrix P′ is calculated based on the remaining 823 sets of data, and a prediction score of the removed binary relation (tⱼ, bₖ) is evaluated.

In general, in the leave-one-out cross-validation, data used as the test sample, that is, data to be removed is all samples in a data set. However, there may be a case where prediction of the binary relation (tⱼ, bₖ) is completely unavailable or the prediction of the binary relation (tⱼ, bₖ) is unnecessary when the certain known ternary relation (tⱼ, bₖ, dₗ) among the treatment method entity tⱼ, the biomarker entity bₖ, and the disease entity dₗ is removed. Therefore, the ternary relation (tⱼ, bₖ, dₗ) corresponding to such cases is excluded from the test sample in the leave-one-out cross-validation in the present embodiment. Hereinafter, an exclusion criterion will be described.

For the certain known ternary relation (tⱼ, bₖ, dₗ) among the treatment method entity tⱼ, the biomarker entity bₖ, and the disease entity dᵢ, when tⱼ or bₖ is absent in the remaining 823 sets of data, the ternary relation (tⱼ, bₖ, dₗ) is excluded from the test sample in the leave-one-out cross-validation. This is because when the ternary relation (tⱼ, bₖ, dₗ) is removed, prediction of the binary relation (tⱼ, bₖ) is completely unavailable.

For the certain known ternary relation (tⱼ, bₖ, dₗ) among the treatment method entity tⱼ, the biomarker entity bₖ, and the disease entity dₗ, when a known ternary relation (tⱼ, bₖ, dm) of a disease dm different from dₗ is present in the remaining 823 sets of data, the ternary relation (tⱼ, bₖ, dₗ) is excluded from the test target in the leave-one-out cross-validation. This is because even when the ternary relation (tⱼ, bₖ, dₗ) is removed, the binary relation (tⱼ, bₖ) is still present in the remaining 823 sets of data.

As a result of applying the exclusion criterion described above, there are 375 sets of ternary relations remaining as the test target in the leave-one-out cross-validation.

As evaluation metrics for prediction accuracy in the leave-one-out cross-validation, a hit rate at N (HR@N) and a detection rate are used.

HR@N represents a proportion of times that the test sample removed in the leave-one-out cross-validation is within top N in ranking of the cross-disease prediction score of the binary relation between the treatment method entity and the biomarker entity. However, only unknown binary relations are contained in the ranking, and the known binary relation is excluded. For example, a statement that “HR@10 is 50%” means that “50% (187 out of 375 sets) of data in the test sample evaluated by the leave-one-out cross-validation has a prediction score ranked within top 10”. Higher HR@N represents a smaller number of predicted false edges (binary relations), that is, higher prediction specificity.

The detection rate represents a proportion of cross-disease prediction scores higher than 0 in the test sample in the leave-one-out cross-validation. For example, a statement that “detection rate is 50%” means that “50% (187 out of 375 sets) of data in the test sample evaluated by the leave-one-out cross-validation has a prediction score higher than 0”. A high detection rate represents high prediction sensitivity.

In the leave-one-out cross-validation, the values of the hyperparameters are set to p=q=u=v=0.25 in calculation of the disease-specific prediction adjacency matrix (Formula 5) according to the proposed method 1 and the proposed method 2. In calculation of the cross-disease prediction adjacency matrix (Formula 9) in the method in related art, the values of the hyperparameters are set to q=v=0.5.

FIG. 14 shows results of HR@N in the proposed method 1, the proposed method 2, and the method in related art. A horizontal axis represents a rank, and a vertical axis represents a hit rate (HR). For example, when the method in related art is used, a data point 1401 indicates that HR@10 is 24.5%.

As shown in FIG. 14, overall, HR@N in the case of using the proposed method 1 and HR@N in the case of using the proposed method 2 are generally equal, both of which tend to be higher than HR@N in the case of using the method in related art. That is, this suggests that the proposed method 1 and the proposed method 2 have higher prediction specificity than that of the method in related art.

In a region 1402 surrounded by a dotted line in FIG. 14, HR rapidly increases relative to an amount of change in the rank. This indicates that the cross-disease prediction score of the test sample is 0, that is, the test sample is at a lowest level. The detection rate in the case of using the proposed method 1 is 73.3%, the detection rate in the case of using the proposed method 2 and the detection rate in the case of using the method in related art are 73.6%. The detection rate in the case of using the proposed method 2 is higher by 0.3% than the detection rate in the case of using the proposed method 1, which suggests that the proposed method 2 has higher prediction sensitivity than that of the proposed method 1.

As described above, the information processing system in the first embodiment or the second embodiment can accurately predict the unknown binary relation between the treatment method and the biomarker.

In the information processing systems according to the first embodiment and the second embodiment, the similarity is calculated based on the number of commonly-connected adjacent treatment method nodes as the inter-node similarity calculation processing (see Formula 1, Formula 2, Formula 7, and Formula 8), and alternatively, another similarity index such as SimRank or a Jaccard index, or a similarity based on node embedding calculated by a machine learning method such as Node2Vec or a graph neural network may be used, or these may be used in combination. Accordingly, it is possible to improve prediction accuracy of the unknown binary relation between the treatment method and the biomarker.

In addition to the treatment method, the biomarker, and the disease, the invention can be extended to a relation of four or more entities by adding another type of entity. For example, a quaternary relation among the treatment method, the biomarker, the disease, and a side effect becomes a ternary relation among the treatment method, the biomarker, and a combination of the disease and the side effect by treating the combination of the disease and the side effect as one entity. In this way, by converting the relation of four or more entities into the ternary relation, the invention can be extended to a relation of four or more entities.

The invention is not limited to the above embodiment, and includes various modifications and equivalent configurations within the scope of the appended claims. For example, the embodiment is described in detail for easy understanding of the invention, and the invention is not necessarily limited to including those all the configurations described above. A part of a configuration of one embodiment can be replaced with a configuration of another embodiment. A configuration of one embodiment can also be added to a configuration of another embodiment. Another configuration may be added to a part of the configuration of an embodiment, and a part of the configuration of each embodiment may be deleted or replaced with another configuration.

A part or all of the above-described configurations, functions, processing units, processing methods, and the like may be implemented by hardware by, for example, designing with an integrated circuit, or may be implemented by software by, for example, a processor interpreting and executing a program for implementing each function.

Information such as a program, a table, and a file for implementing each function can be stored in a storage apparatus such as a memory, a hard disk, or a solid state drive (SSD), or in a recording medium such as an IC card, an SD card, or a DVD.

Control lines and information lines considered to be necessary for description are shown, and not all control lines and information lines necessary for implementation are shown. Actually, it may be considered that almost all the configurations are connected to one another.

What is claimed is:

1. An information processing system for predicting an unknown binary relation between a treatment method and a biomarker based on a known ternary relation among the treatment method, the biomarker, and a disease, the information processing system comprising:
a computer including a computing apparatus that executes predetermined processing and a storage device connected to the computing apparatus, wherein
the information processing system further comprises:
a bipartite graph generation unit that causes the computing apparatus to generate for each disease, based on the known ternary relation, a disease-specific bipartite graph that represents the binary relation between the treatment method and the biomarker;
an inter-node similarity calculation unit that causes the computing apparatus to calculate, based on the disease-specific bipartite graph, a disease-specific inter-treatment-method similarity between treatment methods for the each disease, a cross-disease inter-treatment-method similarity between the treatment methods across all diseases, a disease-specific inter-biomarker similarity between biomarkers for the each disease, and a cross-disease inter-biomarker similarity between the biomarkers across the all diseases;
an unknown edge prediction unit that causes the computing apparatus to calculate at least one of a disease-specific prediction score and a cross-disease prediction score of an unknown edge using the disease-specific bipartite graph, the disease-specific inter-treatment-method similarity, the cross-disease inter-treatment-method similarity, the disease-specific inter-biomarker similarity, and the cross-disease inter-biomarker similarity; and
an output unit that causes the computing apparatus to output at least one of the disease-specific inter-treatment-method similarity, the cross-disease inter-treatment-method similarity, the disease-specific inter-biomarker similarity, the cross-disease inter-biomarker similarity, the disease-specific prediction score, and the cross-disease prediction score.

2. The information processing system according to claim 1, wherein
the bipartite graph generation unit generates a disease-specific adjacency matrix G⁽ⁱ⁾ that represents an edge of the disease-specific bipartite graph, and
the inter-node similarity calculation unit calculates,
based on the disease-specific adjacency matrix G⁽ⁱ⁾, a disease-specific inter-treatment-method similarity matrix T⁽ⁱ⁾ that represents the disease-specific inter-treatment-method similarity,
based on the disease-specific adjacency matrix G⁽ⁱ⁾, a disease-specific inter-biomarker similarity matrix B⁽ⁱ⁾ that represents the disease-specific inter-biomarker similarity,
based on the disease-specific adjacency matrix G⁽ⁱ⁾ and the disease-specific inter-treatment-method similarity matrix T⁽ⁱ⁾, a cross-disease inter-treatment-method similarity matrix T′ that represents the cross-disease inter-treatment-method similarity, and
based on the disease-specific adjacency matrix G⁽ⁱ⁾ and the disease-specific inter-biomarker similarity matrix B⁽ⁱ⁾, a cross-disease inter-biomarker similarity matrix B′ that represents the cross-disease inter-biomarker similarity.

3. The information processing system according to claim 2, wherein
the inter-node similarity calculation unit calculates,
based on node commonality in the disease-specific adjacency matrix G⁽ⁱ⁾, the disease-specific inter-treatment-method similarity matrix T⁽ⁱ⁾,
based on the node commonality in the disease-specific adjacency matrix G⁽ⁱ⁾, the disease-specific inter-biomarker similarity matrix B⁽ⁱ⁾,
based on the node commonality in the disease-specific adjacency matrix G⁽ⁱ⁾ and node commonality in the disease-specific inter-treatment-method similarity matrix T⁽ⁱ⁾, the cross-disease inter-treatment-method similarity matrix T′, and
based on the node commonality in the disease-specific adjacency matrix G⁽ⁱ⁾ and node commonality in the disease-specific inter-biomarker similarity matrix B⁽ⁱ⁾, the cross-disease inter-biomarker similarity matrix B′.

4. The information processing system according to claim 3, wherein
the inter-node similarity calculation unit calculates
the disease-specific inter-treatment-method similarity matrix T⁽ⁱ⁾ in which a similarity increases as the number of commonly-connected nodes in the disease-specific adjacency matrix G⁽ⁱ⁾ increases,
the disease-specific inter-biomarker similarity matrix B⁽ⁱ⁾ in which a similarity increases as the number of commonly-connected nodes in the disease-specific adjacency matrix G⁽ⁱ⁾ increases,
the cross-disease inter-treatment-method similarity matrix T′ in which a similarity increases as the number of nodes in the disease-specific adjacency matrix G⁽ⁱ⁾ and the disease-specific inter-treatment-method similarity matrix T⁽ⁱ⁾ increases, and
the cross-disease inter-biomarker similarity matrix B′ in which a similarity increases as the number of nodes in the disease-specific adjacency matrix G⁽ⁱ⁾ and the disease-specific inter-biomarker similarity matrix B⁽ⁱ⁾ increases.

5. The information processing system according to claim 1, wherein
the bipartite graph generation unit extracts a known binary relation between the treatment method and the biomarker based on the known ternary relation, and generates a cross-disease bipartite graph representing the binary relation between the treatment method and the biomarker, and a cross-disease adjacency matrix G′ representing an edge of the cross-disease bipartite graph, and
the inter-node similarity calculation unit calculates, based on node commonality in the cross-disease adjacency matrix G′, a cross-disease inter-treatment-method similarity matrix T′ and a cross-disease inter-biomarker similarity matrix B′.

6. The information processing system according to claim 2, wherein
the unknown edge prediction unit calculates, using the disease-specific adjacency matrix G⁽ⁱ⁾, the disease-specific inter-treatment-method similarity matrix T⁽ⁱ⁾, the disease-specific inter-biomarker similarity matrix B⁽ⁱ⁾, the cross-disease inter-treatment-method similarity matrix T′, and the cross-disease inter-biomarker similarity matrix B′, a disease-specific prediction adjacency matrix P⁽ⁱ⁾ representing the disease-specific prediction score of a known or unknown relation between the treatment method and the biomarker, and a cross-disease prediction adjacency matrix P′ representing the cross-disease prediction score.

7. The information processing system according to claim 6, wherein
the unknown edge prediction unit calculates, using a sum of a value obtained by multiplying a sum of the disease-specific inter-treatment-method similarity matrix T⁽ⁱ⁾ and the cross-disease inter-treatment-method similarity matrix T′ by the disease-specific adjacency matrix G⁽ⁱ⁾ and a value obtained by multiplying a sum of the disease-specific inter-biomarker similarity matrix B⁽ⁱ⁾ and the cross-disease inter-biomarker similarity matrix B′ by the disease-specific adjacency matrix G⁽ⁱ⁾, the disease-specific prediction adjacency matrix P⁽ⁱ⁾ and the cross-disease prediction adjacency matrix P′.

8. The information processing system according to claim 1, wherein
the output unit outputs the binary relation between the treatment method and the biomarker, which has a higher similarity than a predetermined threshold and a higher prediction score than a predetermined threshold.

9. The information processing system according to claim 1, wherein
the output unit
receives, as an input, a treatment method to be displayed, and
extracts a treatment method whose similarity to the input treatment method is equal to or higher than a predetermined threshold, and outputs the extracted treatment method, or
extracts a biomarker whose prediction score with respect to the input treatment method is equal to or higher than a predetermined threshold, and outputs the extracted biomarker.

10. The information processing system according to claim 1, wherein
the output unit
receives, as an input, a biomarker to be displayed, and
extracts a biomarker whose similarity to the input biomarker is equal to or higher than a predetermined threshold, and outputs the extracted biomarker, or
extracts a treatment method whose prediction score with respect to the input biomarker is equal to or higher than a predetermined threshold, and outputs the extracted treatment method.

11. The information processing system according to claim 1, wherein
the output unit displays, in a table format, the disease-specific inter-treatment-method similarity, the cross-disease inter-treatment-method similarity, the disease-specific inter-biomarker similarity, the cross-disease inter-biomarker similarity, the disease-specific prediction score, or the cross-disease prediction score.

12. The information processing system according to claim 1, wherein
the output unit displays the known binary relation between the treatment method and the biomarker, and the predicted binary relation between the treatment method and the biomarker using at least one of the disease-specific bipartite graph and the cross-disease bipartite graph for the treatment method and the biomarker.

13. A prediction method for predicting, by an information processing system, an unknown binary relation between a treatment method and a biomarker based on a known ternary relation among the treatment method, the biomarker, and a disease, wherein
the information processing system includes a computer including a computing apparatus that executes predetermined processing and a storage device connected to the computing apparatus, and
the prediction method comprising:
a bipartite graph generation procedure of causing the computing apparatus to generate for each disease, based on the known ternary relation, a disease-specific bipartite graph that represents the binary relation between the treatment method and the biomarker;
an inter-node similarity calculation procedure of causing the computing apparatus to calculate, based on the disease-specific bipartite graph, a disease-specific inter-treatment-method similarity between treatment methods for the each disease, a cross-disease inter-treatment-method similarity between the treatment methods across all diseases, a disease-specific inter-biomarker similarity between biomarkers for the each disease, and a cross-disease inter-biomarker similarity between the biomarkers across the all diseases;
an unknown edge prediction procedure of causing the computing apparatus to calculate at least one of a disease-specific prediction score and a cross-disease prediction score of an unknown edge using the disease-specific bipartite graph, the disease-specific inter-treatment-method similarity, the cross-disease inter-treatment-method similarity, the disease-specific inter-biomarker similarity, and the cross-disease inter-biomarker similarity; and
an output procedure of causing the computing apparatus to output at least one of the disease-specific inter-treatment-method similarity, the cross-disease inter-treatment-method similarity, the disease-specific inter-biomarker similarity, the cross-disease inter-biomarker similarity, the disease-specific prediction score, and the cross-disease prediction score.
